(19) United States
(12) Patent Application Publication
(10) Pub. No.: US 2021/0110060 A1
(43) Pub. Date: Apr. 15, 2021

(54) SYSTEM FOR MANAGING MEDICAL DATA

(71) Applicant: Mitchell Sacks, Montauk, NY (US)

(72) Inventor: Mitchell Sacks, Montauk, NY (US)

(73) Assignee: Mesa Partners, Inc., Montauk, NY (US)

(21) Appl. No.: 16/817,803

(22) Filed: Mar. 13, 2020

Related U.S. Application Data
(60) Provisional application No. 62/915,213, filed on Oct. 15, 2019.

(51) Int. Cl. G06F 21/62; G16H 10/00; G16H 40/20; G06F 21/60; H04L 9/32

(52) U.S. Cl. CPC G06F 21/6245; G16H 10/00; H04L 9/3228; G06F 21/602; H04L 9/3231; G16H 40/20

(57) ABSTRACT

A system for managing medical data is disclosed, comprising a computing device to permit a user to upload a plurality of medical data files. A server comprises a server engine having an encryption module to encrypt the uploaded plurality of medical data files and store the plurality of encrypted medical data files in a database.

### CROSS-REFERENCE TO RELATED APPLICATIONS

The present application claims priority to U.S. Provisional Application No. 62/915,213 filed on Oct. 15, 2019, entitled “SYSTEM FOR MANAGING MEDICAL DATA” the entire disclosure of which is incorporated by reference herein.

### TECHNICAL FIELD

The embodiments generally relate to electronic systems for managing medical data.

### BACKGROUND

Electronic medical file systems, also known as telemedicine systems, allow medical practitioners to engage in diagnostic activities in a remote location from the patient. Medical practitioners can utilize the telemedicine systems to send and receive medical data without requiring in-person medical appointments.

Although medical data systems are available, there are significant problems that prevent them from being widely implemented. One particular problem is that many medical data systems are unable to be controlled in compliance with HIPAA. Thus, it is possible for medical files to be lost, corrupted, modified, or breached, resulting in significant legal liability.

Other significant problems with medical data systems are the inability to verify the transmitting or receiving physician. Further, many systems do not verify that the receiving system can upload each medical data file or view the file due to different encryption protocols.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments disclosed herein provide for a system for managing medical data, comprising a computing device to permit a user to upload a plurality of medical data files. A server includes an integrated server engine having an encryption module to encrypt the uploaded medical data files and store the plurality of encrypted medical data files in a database. A verified user may then provide access for themselves or temporary access to another person to view the medical data files through a web browser for a predetermined period of time before access to the file viewer is automatically revoked.

The system allows for a user's identity to be verified by the system to allow the user to view medical data files for a pre-determined period of time. The user will be able to engage with the one or more of the plurality of their own medical data files, such as by viewing the medical data file, editing the medical data file, adding a new medical data file, or other means of engaging with the stored and encrypted medical data files within the server.

In one aspect, the server engine further comprises a verification module to verify the identity of the user.

In one aspect, when a user registers a new user id for storing medical records an encrypted authorization key is generated on the device where the account was created, and a session token is created on the server for the new user id.

In one aspect, the application utilizes biometrics authentication to verify the user where biometrics are available.

In one aspect, when a user id signs on a device does not have a matching authorization key, the user will be required to enter an additional time sensitive Login Code which can be retrieved through an email triggered from the login page of the application. This email is sent to the email address associated with the original registration.

In one aspect, a viewing module uses a web browser to permit user or a viewer to view one or more of the plurality of medical data files for a predetermined time period.

In one aspect, when the user provides temporary access to another user, a URL is generated along with a separate 6 digit Keypass. The URL contains a time stamped encrypted key which will only be valid for a limited time and requires the viewer to type in the Keypass before they can view the content.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the network infrastructure of the system, according to some embodiments;

FIG. 2 illustrates a block diagram of the server engine and associated modules, according to some embodiments;

FIG. 3 illustrates a flowchart of a method for managing medical data, according to some embodiments;

FIG. 4 illustrates a block diagram of the viewer time stamp based authentication system, according to some embodiments;

FIG. 5 illustrates a schematic of the medical data file system, according to some embodiments; and

FIG. 6 illustrates a flowchart of the system and method for validating user identity, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments described herein relate to a system for managing medical data over a network to allow for a patient to upload their medical history, which is stored in a secure HIPAA compliant database. The system is accessible on a mobile application or using a desktop computer or similar computing device to allow physicians or other care providers to securely view the patient's medical records.

As used herein, the term “user” may refer to a patient or other person(s) who may store or provide patient medical records and other forms of medical data.

As used herein, the term “viewer” may refer to the patient, practitioner, care giver or other persons(s) who may view the user's medical records for a limited time period.

The embodiments described herein may refer to medical data files or other data types stored within an encrypted server. The embodiments may be particularly useful for industries which utilize secure data storage systems. One skilled in the arts will readily understand that various forms of data can be securely transmitted, stored, and engaged with using the system as described herein.

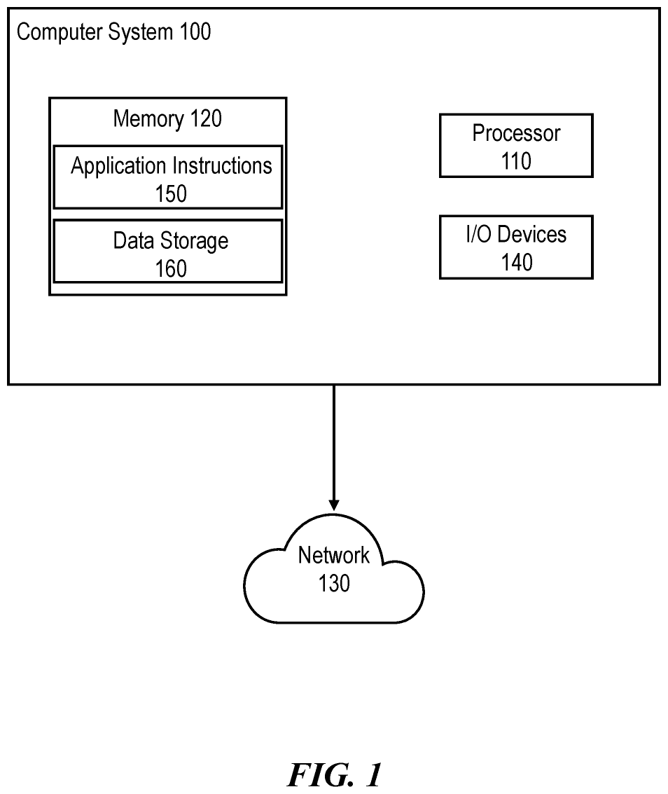

FIG. 1 illustrates a computer system 100, which may be utilized to execute the processes described herein. The computing system 100 is comprised of a standalone computer or mobile computing device, a mainframe computer system, a workstation, a network computer, a desktop computer, a laptop, or the like. The computer system 100 includes one or more processors 110 coupled to a memory 120 via an input/output (I/O) interface. Computer system 100 may further include a network interface to communicate with the network 130. One or more input/output (I/O) devices 140, such as video device(s) (e.g., a camera), audio device(s), and display(s) are in operable communication with the computer system 100. In some embodiments, similar I/O devices 140 may be separate from computer system 100 and may interact with one or more nodes of the computer system 100 through a wired or wireless connection, such as over a network interface.

Processors 110 suitable for the execution of a computer program include both general and special purpose microprocessors and any one or more processors of any digital computing device. The processor 110 will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 100 and other devices attached to a network 130, such as other computer systems, or between nodes of the computer system 100. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

The memory 120 may include application instructions 150, configured to implement certain embodiments described herein, and a database 160, comprising various data accessible by the application instructions 150. In one embodiment, the application instructions 150 may include software elements corresponding to one or more of the various embodiments described herein. For example, application instructions 150 may be implemented in various embodiments using any desired programming language, scripting language, or combination of programming languages and/or scripting languages (e.g., C, C++, C#, JAVA®, JAVASCRIPT®, PERL®, etc.).

The steps and actions of the computer system 100 described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a web site, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

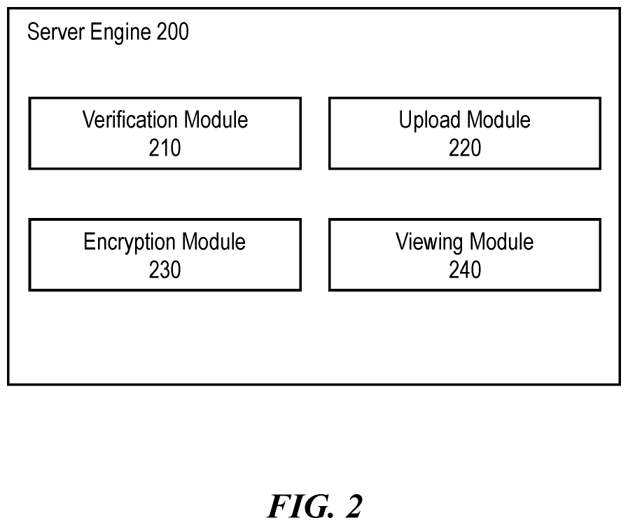

FIG. 2 illustrates a block diagram of the server engine 200 and associated modules to allow for the secure generation, transmission, and viewing of medical data. A verification module 210 receives user login credentials to verify the identity of the user. The verification module 210 may utilize various forms of user authentication, including usernames, passwords, biometrics, and two-factor authentication among other secure verification means known in the arts. An upload module 220 permits the user to upload medical files or other data to the system such that an encryption module 230 may encrypt the data for transmissions and storage within the system. A viewing module 240 allows the user to view medical data in a browser window. For example, the viewing module 240 may allow the physician to view the medical data for a predetermined period of time, such as for one hour, to minimize the risk of a data breach. The encryption module 230 may also operate to decrypt the data to allow the viewing module 240 to display the medical data to the physician.

In some embodiments, biometric identification is requested by the system to access the application. The user must successfully complete biometric identification and authentication via a username and password which is encrypted using a secure key combined with the username to produce a unique encryption for each user. The system may then use a secure encrypted connection to the server to deliver the username and encrypted password to a lambda function which verifies that the encrypted key matches the stored version on the server. If the encrypted password matches the stored password version an access token thirty-two characters in length is returned to the application. The access token is stored with the username and password in the user table, along with an expiration time. This access token will continue to be used when the application communicated with the server to upload files and is incorporated into temporary access keys used to invite doctors.

In some embodiments, files are encrypted via the encryption module 240 and remain encrypted while stored in the database to prevent the risk of a data breach. This allows for the control of the medical records to reduce the risk of data being released to the public domain or other hacking mechanism.

The system allows medical record data files to be tracked and for access to such medical record data files or medical record data contained within such medical record data files to be tracked, coordinated and controlled so as to prevent unauthorized modification of data, to maintain the integrity of the data file, and to ensure continuity of the medical record data file. The system thus allows medical data services and other suitable services requiring access to a medical record data file to be provided without incurring the risks posed by prior art systems.

In some embodiments, users may upload medical data files and selected any desired information such as physician name, location, organ system, and external storage source for the medical data files to upload. The selected medical data file is encoded/decoded into base64 and sent to a lambda function along with the access token and the meta data. The access token is then compared with the one stored in the user table. If the access token are not matched, upload is immediately terminated and an error notification is returned. The medical data files are then stored in the records table and the files is stored in the s3 table with encryption while a successful response notification is returned.

The user may access a record list, profile, registration, or uploaded record. Generating a time stamped access key generates an encrypted message which uses the access token for the user in addition to the current time and a time span to generate a time stamp which will remain valid for a predetermined time period (such as 5 minutes). This short-term key is generated in the same process as described above. However, the time span may be shortened, or otherwise altered. The payload is processed by a single lambda function smmrauth. This function is the only path to retrieve uploaded files, file lists, or other user submitted information stored on the lambda server. The smmrauth function receives the encrypted key along with a field used for identifying the desired data. When called by the application the encryption system uses a keypass set to “000000” and a short access period. Using this keypass the information can be retrieved immediately. After verifying the encrypted time stamped key, the function will pull the data out of the S3 table, decrypt the data, then return it to the caller in base 64 over an SSL connection. If the medical data file refers to a .pdf, .jpg, or other supported image format, the display is handled by the same web page generating system used by the doctor page. No file is returned. Instead, the base 64 data for the files is used to populate a javascript Blob in a dynamically generated web page.

In some embodiments, to provide physician access to the system a time stamped encrypted key is generated using the current time but incorporating a user-selected period of time such as 10 minutes, 30 minutes, 1 hour, or 2 hours. A six-digit keypass is displayed at the point the access link is to be sent to the doctor. The keypass is not included in the link that is sent to the doctor and the link will not function properly without the link. The application can send a link by generating an email or by using the share menu or SMS depending on the tools available on the platform that the application is being run on. In addition, the user can send a separate message containing the keypass. The emailed link will open a web page allowing the user(s) records to be accessed for a limited time. When the physician accesses the link, if they have not entered the keypass yet, they are prompted to enter the 6-digit value. Until the 6-digit value is entered and confirmed the physician will be unable to access any information. Once the keypass is entered it will not need to be entered again until the web page is closed, but any new browser windows will need to have the keypass entered in order to progress. Each individual record, list of records, or user entered registration and profile information is retrieved using the smmrauth lambda function passing in the generated time stamped message and the keypass the doctor enters. When the valid time period for the time stamped message has expired, the smmrauth lambda function will no longer provide access to any information.

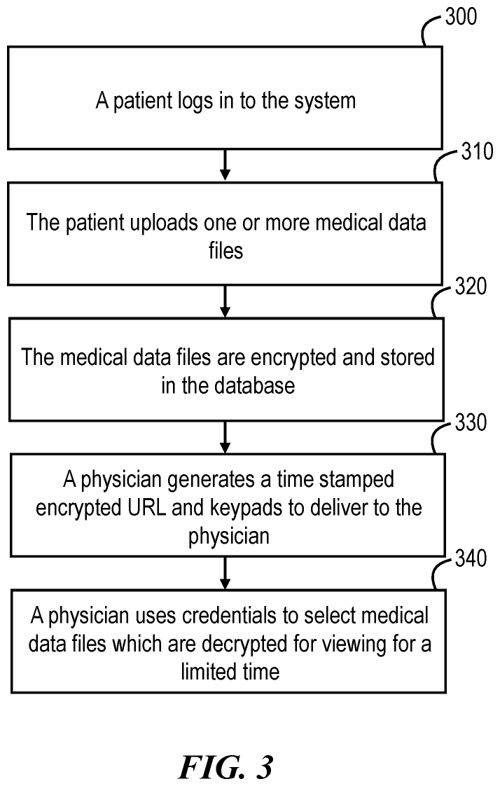

FIG. 3 illustrates a flowchart of a method for securely uploading, transmitting, and storing medical data using the system provided hereinabove. In step 300, a patient logs in to the system via a verification module, and user permissions are determined via a permissions module. If the user is a patient, the patient may upload one or more medical data files, such as their patient medical history, in step 310. In step 320, the one or more medical data files are encrypted by an encryption module to allow for the secure transfer of data through the system to the secure database. In step 330, the patient generates a time stamped encrypted url and keypass to be delivered electronically to the physician. In step 340, A physician selects a medical data file to view, the encryption module decrypts the data and the physician is able to view the medical data file via the viewing module.

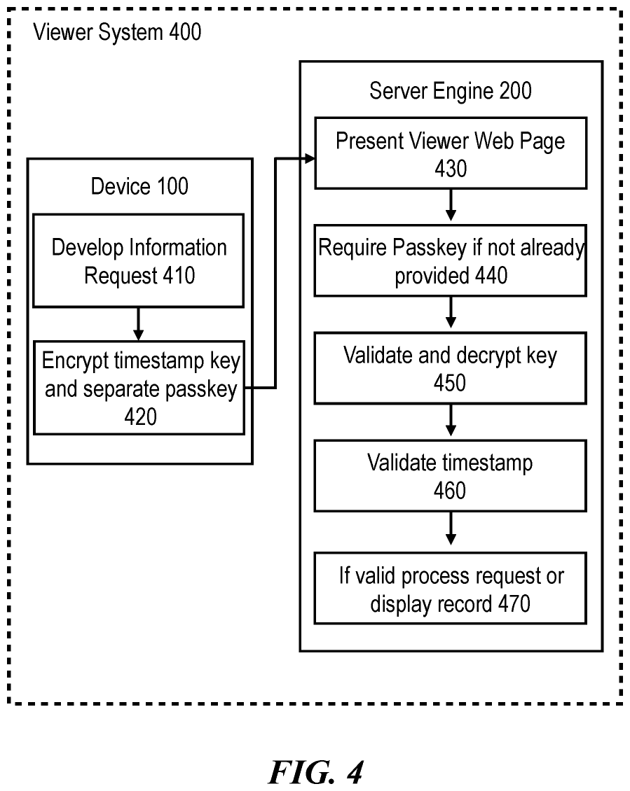

FIG. 4 illustrates a block diagram of the viewer system time stamped record access system. Device 100 develops a request 410 for the Server Engine 200. A time stamped encrypted key and passkey (420) is generated which will be passed to the Viewer System to present a web page interface (430). If a passkey has not been delivered a dialog box requests entry (440), otherwise the engine decrypts the timestamped key 450 and validates that it is still valid (460). If all is successful, process the request 470 which may be to return file information or display a catalog of the multiple files.

In some embodiments, the biometric device may be provided as a standalone device, such as a hand or finger scanner, facial recognition scanner, or other biometric scanning device known in the art. Similarly, the biometric device may be provided with a computing device, such as a smartphone which has finger scanning, facial recognition or similar capabilities.

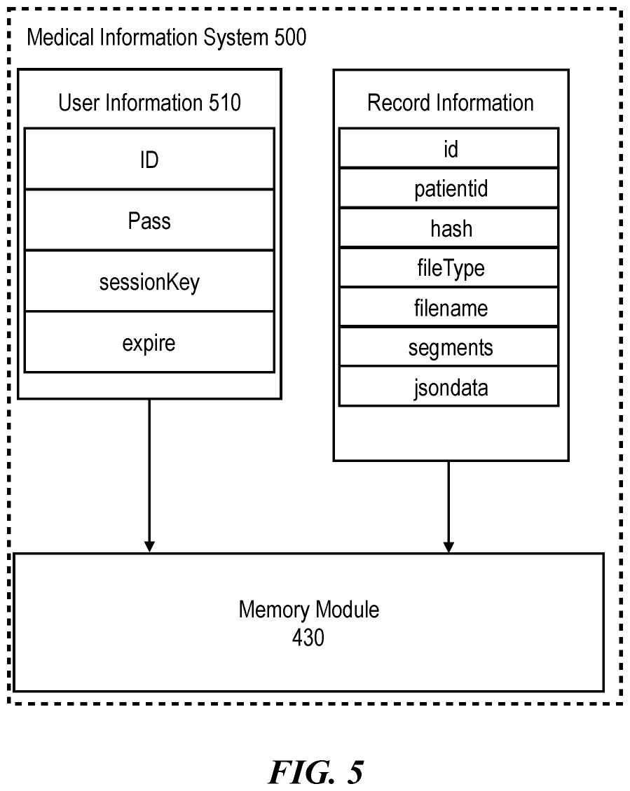

FIG. 5 illustrates a schematic of the medical file system 500 comprising a plurality of medical information 510 comprising user information used for validating access to the record information 520 (e.g., patient ID, patient name, patient address, patient insurance, attending physician, clinical data, lab results and other information known in the arts) stored in a memory module 430. Medical information is encrypted as described hereinabove to provide security to the system.

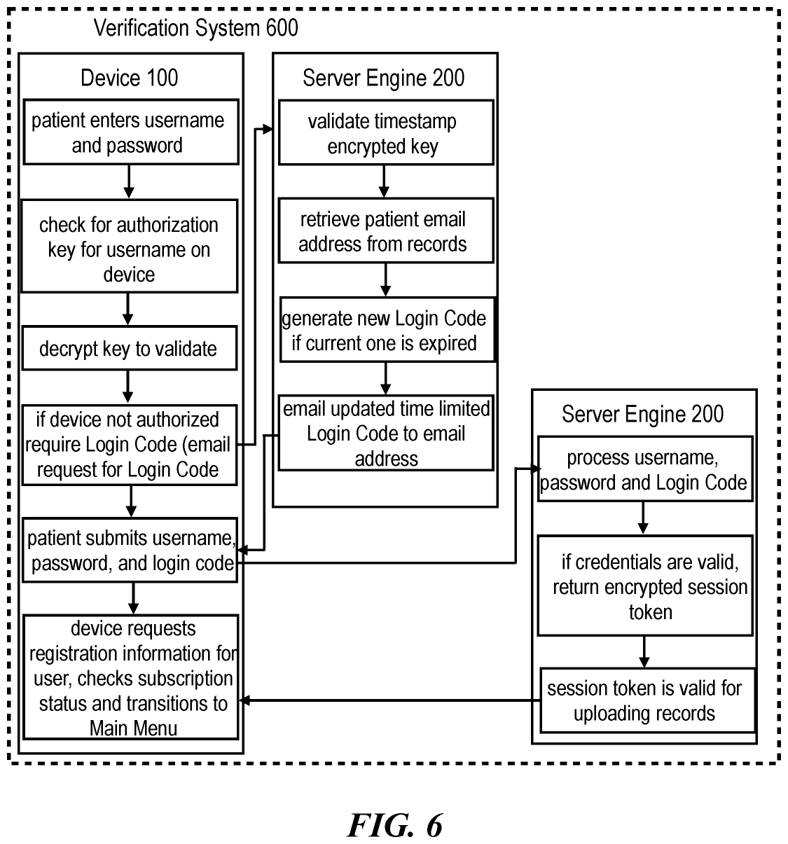

FIG. 6 illustrates the system used to validate user identity. The patient enters a username and password and the system checks for the authorization key for the username on the device 100. The key is then decrypted. If the device is not authorized, a login code is required via an email request for the login code. The patient may then submit a username, password and login code. The device then requests registration information for the user, checks the subscription status of the user, and transitions to the main menu interface. The server engine 200 validated the timestamp encrypted key and retrieves the patient email and address from the records. A new login code is generated if the current login code has expired. Next, the updated time-limited login code is emailed. The server engine may also process username, password and login code and if the credentials are valid, return an encrypted session token.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for anyone of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A system for managing medical data, the system comprising:
a computing device to permit one or more users to upload a plurality of medical data files; and
a server comprising a server engine having an encryption module to encrypt the uploaded plurality of medical data files and store the plurality of encrypted medical data files in a database, the database read/write accessible by the user, and readable by viewers using time stamped keys.

2. The system of claim 1, wherein server engine further comprises a verification module to verify the identity of the user.

3. The system of claim 2, wherein the verification module only allows access from devices authorized through email verification Login Code.

4. The system of claim 2, wherein the verification module utilizes biometrics to verify the user.

5. The system of claim 4, wherein a viewing module permits the user to view one or more of the plurality of medical data files for a predetermined time period.

6. The system of claim 5, wherein the one or more of the plurality of medical data files are encrypted after the predetermined time period has elapsed. A system for managing medical data, the system comprising:
a computing device to permit a user to upload a plurality of medical data files; and
a server comprising a server engine having an encryption module to encrypt the uploaded plurality of medical data files and store the plurality of encrypted medical data files in a database; and
a viewing module to permit a user to view at least one of the plurality of encrypted medical data files for a pre-determined time period, wherein the pre-determined time period is determined by a user permissions module.

8. The system of claim 7, wherein server engine further comprises a verification module to verify the identity of the user.

9. The system of claim 7, wherein the verification module utilizes two-factor authentication to verify the user.

10. The system of claim 2, wherein the verification module utilizes biometric information to verify the user.

11. The system of claim 10, wherein the biometric information is captured via a biometric device.

12. The system of claim 11, wherein a comparator compares biometric information received via the biometric device and compares the received biometric information to a plurality of stored biometric information stored in a memory module to determine the identity of the user.

13. The system of claim 13, wherein a viewing module permits the user to view one or more of the plurality of medical data files for a predetermined time period.

14. A method for managing medical data, the system comprising:
uploading, via a computing device in communication with a database via a network, a plurality of medical data files;
encrypting, via an encryption module, the plurality of medical data files and transmitting the plurality of encrypted medical data files to the database;
decrypting at least one of the plurality of medical data files and displaying the at least one of the plurality of medical data files on a user interface of the computing device

15. The method of claim 15, wherein a server engine comprises a verification module to verify the identity of the user.

16. The method of claim 16, wherein the verification module utilizes two-factor authentication to verify the user.

17. The method of claim 16, wherein the verification module utilizes biometrics to verify the user.

18. The method of claim 16, wherein a viewing module permits the user to view one or more of the plurality of medical data files for a predetermined time period.
